(19) United States
(12) Patent Application Publication
(10) Pub. No.: US 2024/0165417 A1
(43) Pub. Date: May 23, 2024

(54) Cell Phone Defibrillator

(71) Applicant: Catricia Roberson, Bowie, MD (US)

(72) Inventor: Catricia Roberson, Bowie, MD (US)

(21) Appl. No.: 18/058,024

(22) Filed: Nov. 22, 2022

(51) Int. Cl. A61N 1/39; H04M 1/21

(52) U.S. Cl. CPC A61N 1/3904; H04M 1/21; A61N 1/046

(57) ABSTRACT

An article of manufacture for providing a cell phone defibrillator according to the present invention includes a mobile application executing instructions on a processor of a smartphone, a charging circuit, and a pair of adhesive electrodes coupled to a charge triggering switch and a charge storage device. The charging circuit includes a control sequencer, a power source, a charge storage device, a charge triggering switch coupled to the charge storage device, a communications interface communicatively coupling the control sequencer to the smartphone, a charge driver coupled between the power source and the charge storage device, a charge sensor coupled between the charge storage device and the control sequencer, and a signal interface coupled between the charge triggering switch and the control sequencer.

### TECHNICAL FIELD

This application relates in general to an article of manufacture for providing life saving devices, and more specifically, to an article of manufacture providing a cell phone defibrillator.

### BACKGROUND

An automated external defibrillator (AED) has been shown to provide life saving treatment to individuals suffering cardiac problems. The benefits of having AEDs available has led to the installation of easy to operate AEDs into many public and private facilities for use when needed. The AEDs themselves have been automated and provided simple instructions in the form of picture graphics on how to properly use the devices. Even with these developments, individuals are still suffering cardiac problems that may lead to death and/or disabilities because an AED is not available when needed.

Therefore, a need exists for an article of manufacture for providing a cell phone defibrillator. The present invention attempts to address the limitations and deficiencies in prior solutions according to the principles and example embodiments disclosed herein.

### SUMMARY

In accordance with the present invention, the above and other problems are solved by providing an article of manufacture providing a cell phone defibrillator according to the principles and example embodiments disclosed herein.

In one embodiment, the present invention is an article of manufacture for providing a cell phone defibrillator. The cell phone defibrillator includes a mobile application executing instructions on a processor of a smartphone, a charging circuit, and a pair of adhesive electrodes coupled to a charge triggering switch and a charge storage device. The charging circuit includes a control sequencer, a power source, a charge storage device, a charge triggering switch coupled to the charge storage device, a communications interface communicatively coupling the control sequencer to the smartphone, a charge driver coupled between the power source and the charge storage device, a charge sensor coupled between the charge storage device and the control sequencer, and a signal interface coupled between the charge triggering switch and the control sequencer.

In another aspect of the present invention, the communications interface communicatively coupling the control sequencer to the smartphone over an external cable.

In another aspect of the present invention, the communications interface communicatively coupling the control sequencer to the smartphone over a wireless communications channel.

In another aspect of the present invention, the charge storage device is a capacitor.

In another aspect of the present invention, the closing of the charge triggering switch discharges the charge storage device across the pair of adhesive electrodes.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of an article of manufacture providing a cell phone defibrillator according to the present invention.

FIG. 2 illustrates another article of manufacture providing a cell phone defibrillator according to the present invention.

FIGS. 3a-b illustrate a circuit diagram for an article of manufacture providing a cell phone defibrillator according to the present invention.

FIG. 4 illustrates a computing system of software components for providing a cell phone defibrillator according to the present invention.

FIG. 5 illustrates a flowchart of a process flow performed by software components in an article of manufacture providing a cell phone defibrillator according to the present invention.

FIG. 6 illustrates a generalized schematic of a programmable processing system utilized as the various computing components described herein used to implement an embodiment of the present invention.

### DETAILED DESCRIPTION

This application relates in general to an article of manufacture for providing life saving devices, and more specifically, to an article of manufacture providing a cell phone defibrillator according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “individual,” and “user” refer to an entity, e.g., a human, using an article of manufacture providing a cell phone defibrillator according to the present invention. The term user herein refers to one or more users.

The term “invention” or “present invention” refers to the invention being applied for via the patent application with the title “Cell Phone Defibrillator.” Invention may be used interchangeably with defibrillator.

The term “cell phone” or “smartphone” refers to a mobile computing device having network communications with telephone, cellular, and Internet-based networks. The mobile computing devices may perform functions implemented and executed by mobile applications controlling internal devices and components and external devices that are communicatively coupled to the smartphones. The terms cell phone, smartphone, and mobile computing device may be used interchangeably.

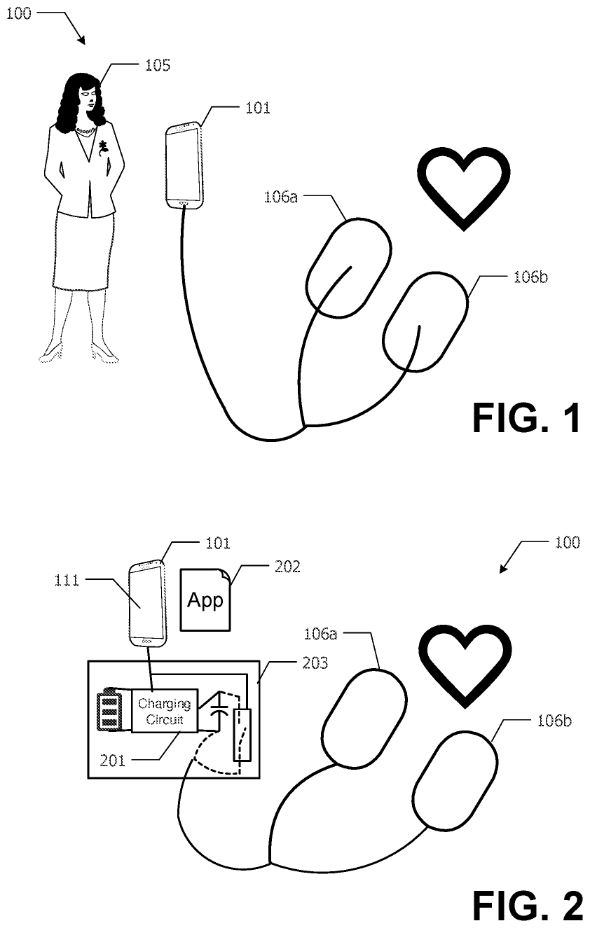

In general, the present disclosure relates an article of manufacture providing a cell phone defibrillator according to the present invention. To better understand the present invention, FIG. 1 illustrates an example embodiment of an article of manufacture providing a cell phone defibrillator according to the present invention. Cell phones or smartphones 101 have become an essential device that is being carried by a significant portion of the public. The cell phones 101 are programmable computing devices that may execute a plurality of applications 202 to perform a wide variety of functions. These applications 202 may be readily downloaded from online app stores such Apple™ App Store and Google Play™.

Applications 202 may control all components within the smartphones 101 as well as devices that are communicatively coupled to the smartphones 101. External devices may be coupled to the smartphone 101 using an external cable inserted into a communications port and/or using a wireless communications channel. The wireless communications may utilize any wireless protocol and technology including infrared data links, Bluetooth™ and related RF data links, WiFi and cellular networks including 3G, 4G LTE, and 5G.

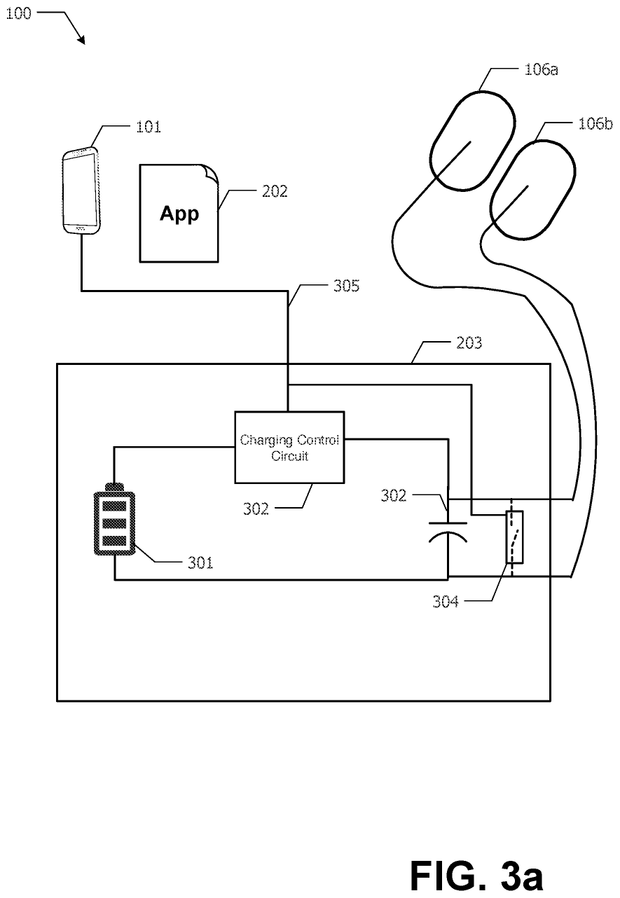

FIG. 2 illustrates another article of manufacture providing a cell phone defibrillator according to the present invention. The defibrillator 100 may utilize a smartphone 101 to control its operation with adhesive electrodes 106a-b electrically coupled to the smartphone 101. The defibrillator 100 comprises a charging circuit 201 that provides electrical charge to the adhesive electrodes 106a-b, a power source such as battery, and control functions provided by the application 202 executing within the smartphone 101. The charging circuit 201 coupled to the adhesive electrodes 106a-b and the power source may be contained within the smartphone 101 and/or may be contained within an external enclosure 203 that is electrically coupled to the smartphone 101. In an alternate embodiment, the charging circuit 201 and the power source may be contained within the smartphone 101 having the adhesive electrodes 106a-b coupled thereto. The charging circuit 201 uses electrical energy from the power source to generate an electrical charge across the adhesive electrodes 106a-b once the adhesive electrodes 106a-b are attached to a patient. The application 202 running on the smartphone 101 controls the charging circuit 201 to set any parameters of the electrical charge applied to the adhesive electrodes 106a-b as well as the timing of the electrical charge.

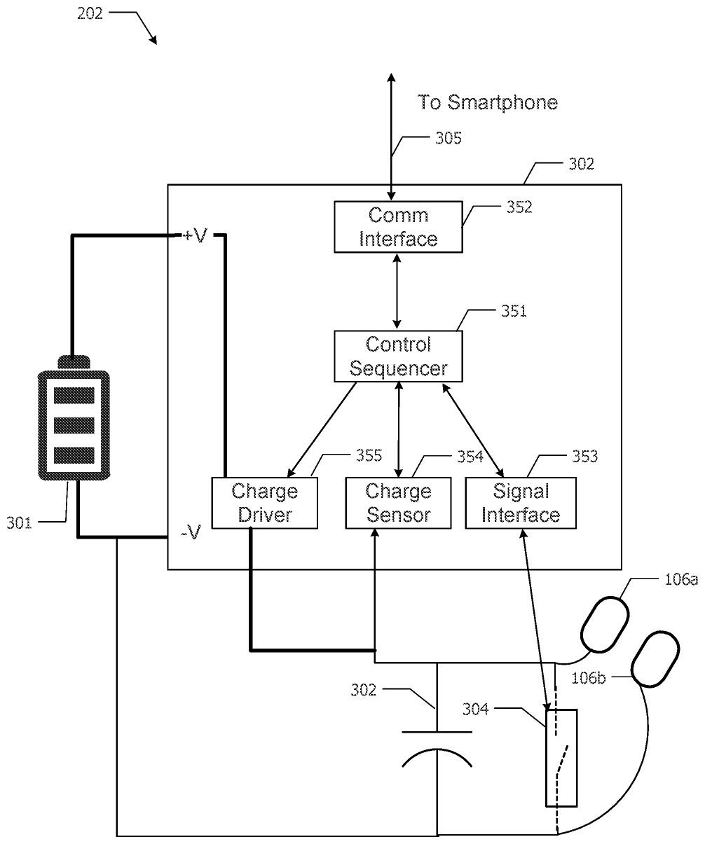

FIGS. 3a-b illustrate a circuit diagram for an article of manufacture providing a cell phone defibrillator according to the present invention. The charging circuit 201 comprises a battery as a power source 301, a charging control circuit 302, a charge storage device 303, and a charge triggering switch 304. The charging circuit 201 is electrically coupled to the smartphone 101 by an external cable 305 in the embodiment of FIG. 3. The smartphone 101 provides commands to the charging control circuit 302 that are generated by the application 202 to operating the defibrillator 100.

The battery 301 acts as a power source to power the defibrillator 100 and its components. The battery 301 also provides electric charge that is provided across the adhesive electrodes 106a-b when the defibrillator 100 is activated. The battery 301 may also be any other mobile power source such a power cells, solar cells and capacitors, and similar devices that are capable of storing electrical energy that may be used by the defibrillator 100 to operate.

The charging control circuit 302 receives commands from the application 202 in the smartphone 101 and enables the operation of the components of the charging circuit 201 in response to the commands. The charging control circuit 302 also sends messages to the application 202 in the smartphone 101 regarding the operation of the components in the charging circuit 201 including charge levels in the battery 301 and the charge storage device 303. The charging control circuit 302 is described in more detail in FIG. 3b.

The charge storage device 303 is a device such as a capacitor that may accumulate electrical energy to generate an electrical charge across the adhesive electrodes 106a-b at a desired voltage. The charging control circuit 302 enables the charge storage device 303 to receive electrical energy from the battery 301 until the charge storage device 303 contains the electrical energy to generate an electrical charge across the adhesive electrodes 106a-b at a desired voltage. The charge storage device 303 rapidly discharges any electrical energy stored therein as a voltage across the adhesive electrodes 106a-b upon the closing of the charge triggering switch 304.

The charge triggering switch 304 is a controllable switch that opens and closes upon receipt of commands from the charging control circuit 302. The charge triggering switch 304 electrically couples the charge storage device 303 to the adhesive electrodes 106a-b when the controllable switch is closed. The charge triggering switch 304 is open by default and remains upon while the charge storage device 303 is accumulating electrical energy.

FIG. 3b illustrates a circuit diagram for the charging control circuit 303 in the charging circuit of the article of manufacture providing a cell phone defibrillator according to the present invention. The charging control circuit 303 comprises a control sequencer 351, a communications interface 352, a signal interface 353, a charge sensor 354, and a charge driver 355.

The control sequencer 351 is a control device that causes the charging control circuit 302 to enable and disable charging of the charge storage device 303 and to open and close the charge triggering switch 304 to discharge the voltage across the adhesive electrodes 106a-b. The control sequencer 351 may be constructed using a programmable processor and may also be constructed using a state machine-based sequencer in which the charging of the charge storage device 303 and the opening and closing the charge triggering switch 304 are controlled.

The communications interface 352 comprises components to enable communications between the smartphone 101 and the control sequencer 351. As noted herein, the communications data link between the smartphone 101 and the control sequencer 351 may utilize an external cable 305 and/or a wireless data link. The communications interface 352 provides the electrical components to establish the data link between the control sequencer 351 and the smartphone whether it is a cable connection using a supported protocol which may include a USB-C connection and a lightening cable connection. The communications interface 352 may also comprise one or more wireless communication transceivers (not shown) to support a wireless connection to the smartphone 101.

The signal interface 353 comprises circuit components to transmit control signals from the control sequencer 352 to the components of the charging circuit 201. The signal interface 353 also receives and amplifies any signals from the charge storage device 303 and the adhesive electrodes 106a-b to enable the signals to be received and used by the control sequencer 351.

The charge sensor 354 is an electrical component that provides the control sequencer 351 with an electrical signal that corresponds to the charge level stored within the charge storage device 303. The charge sensor 354 may generate an analog signal for the current charge level. The charge sensor 354 may also use an analog-to-digital converter (not shown) to encode a charge level into a digital value to be communicated to the application 202.

The charge driver 355 is an electrical component that provides electrical charge from the battery 301 to the charge storage device 303. The charge driver 355 may comprise a driver component capable of sending and/or sinking a required amount of electrical current needed during the charging process. The charge driver 355 also enables and disables the charging of the charge storage device 303 upon command from the control sequencer 351.

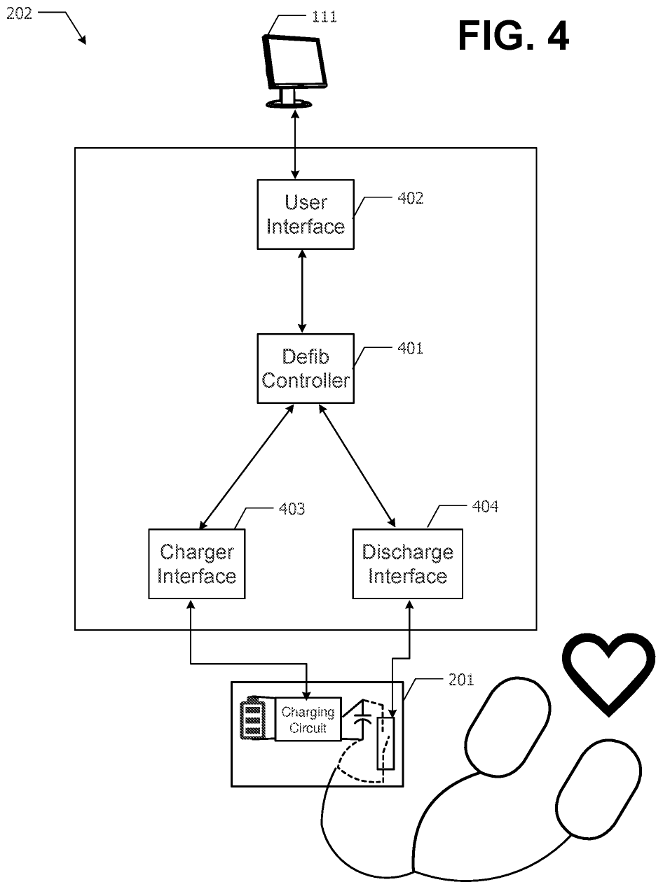

FIG. 4 illustrates a computing system of software components for providing a cell phone defibrillator according to the present invention. The application 202 comprises a set of software components. These software components comprise a defibrillator controller 401, a user interface 402, a charger interface 403, and a discharge interface 404. The charger interface 403 and the discharge interface 404 are communicatively coupled to the charging circuit 201.

The defibrillator controller 401 controls the operation of the application 202. The defibrillator controller 501 sends images and messages to the user via the touch screen 111 of the smartphone 101 using the user interface 402. The defibrillator controller 501 also receives input messages from the user via the touch screen 111 of the smartphone 101 using the user interface 402. The defibrillator controller 501 sends and receives signals and messages with the control sequencer 351 using the charger interface 403 and the discharger interface 404 to charge and discharge the charge storage device 303.

The user interface 402 provides input and output processing to provide a driver with messages and data needed to operate the defibrillator 100. This user interface module 402 also accepts commands from the user to instruct the application 202 to perform these tasks.

The charger interface 403 sends and receives messages with the control sequencer 351 to control the charging of the charge storage device 303. The charger interface 403 also receives and processes the signals from the charge sensor 354, which may include analog and/or digitally encoded signals, as needed by the application.

The discharge interface 404 sends and receives messages with the control sequencer 351 to control the opening and closing the charge triggering switch 304.

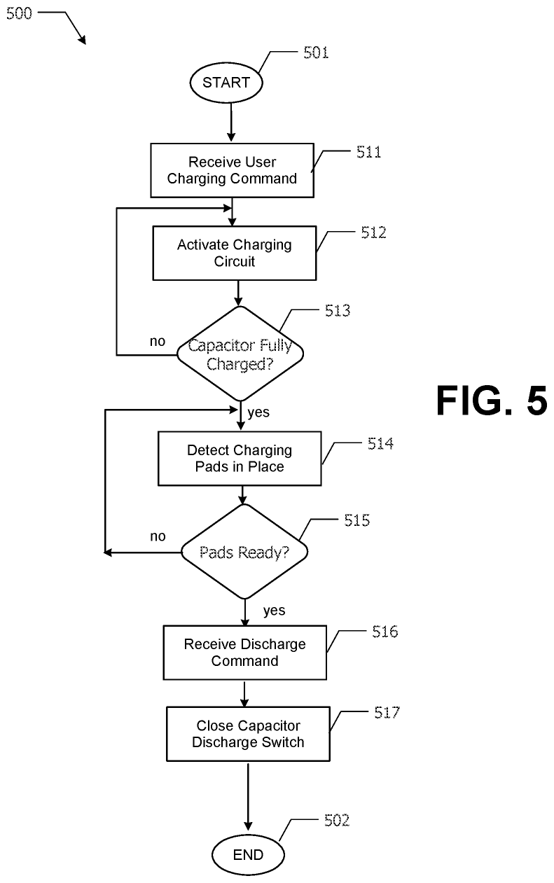

FIG. 5 illustrates a flowchart of a process flow 500 performed by software components in an article of manufacture providing a cell phone defibrillator according to the present invention. Process flow 500 begins 501 when the application 202 receives a user charging command via the user interface 402 in step 511. In step 512, the application 202 transmits an activate charging command to the charging control circuit 301 to activate the charging of charge storage device 303. Test step 513 determines whether the charge level within the charge storage device 303 has reached a desired voltage, and if not, the process 500 returns to step 512 to continue charging the charge storage device 303. The application 202 may request and receive the charge level of the charge storage device 303 from the charge sensor 354 via the control sequencer 351 for use in the determination of test step 513.

When the test step 513 determines that the charge level of the charge storage device 303 has reached a desired voltage, the process flow 500 continues to step 514 in which the application receives confirmation that the adhesive electrodes 106a-b are in place. The application 202 may receive confirmation by providing a query to the user of the defibrillator 100 on the touch screen 111 of the smartphone 101. The user may input confirmation to a button or similar screen control including a swipe on a screen control item when the adhesive electrodes 106a-b are secured in place. The application 202 may also output to the touch screen instructions and an installation diagram for use by the user to properly place the adhesive electrodes onto the patient.

The application 202 may also determine that the adhesive electrodes 106a-b are in place from a signal generated by the charging control circuit 302. The charging control circuit 302 may detect an impedance between the adhesive electrodes 106a-b that indicates the adhesive electrodes 106a-b are attached to the patient. Test step 515 may use either or both of these confirmation indications to determine whether the defibrillator 100 is configured for use. When test step 515 determines the defibrillator 100 is properly configured, the process flow 500 proceeds to step 516, otherwise, the process flow 500 returns to step 514 until the adhesive electrodes 106a-b are in place.

In Step 516, the application 202 receives a discharge command from the user via the touch screen 111 of the smartphone 101. The application 202 commands the charge control circuit 302 to close the charge triggering switch 304 to discharge the electrical voltage in the charge storage device 303 across the adhesive electrodes 106a-b. Once the electrical voltage in the charge storage device 303, the process flow 500 ends 502.

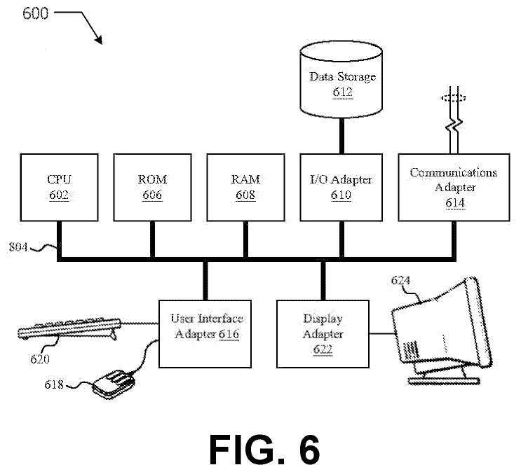

FIG. 6 illustrates a generalized schematic of a programmable processing system utilized as the various computing components described herein used to implement an embodiment of the present invention. FIG. 6 illustrates a computer system 600 adapted according to certain embodiments of the server and/or the user interface device. The central processing unit (“CPU”) 602 is coupled to the system bus 604. The CPU 602 may be a general purpose CPU or microprocessor, graphics processing unit (“GPU”), and/or microcontroller. The present embodiments are not restricted by the architecture of the CPU 602 so long as the CPU 602, whether directly or indirectly, supports the operations as described herein. The CPU 602 may execute the various logical instructions according to the present embodiments.

The computer system 600 also may include random access memory (RAM) 608, which may be synchronous RAM (SRAM), dynamic RAM (DRAM), synchronous dynamic RAM (SDRAM), or the like. The computer system 600 may utilize RAM 608 to store the various data structures used by a software application. The computer system 600 may also include read only memory (ROM) 606 which may be PROM, EPROM, EEPROM, optical storage, or the like. The ROM may store configuration information for booting the computer system 600. The RAM 608 and the ROM 606 hold user and system data, and both the RAM 608 and the ROM 606 may be randomly accessed.

The computer system 600 may also include an input/output (I/O) adapter 610, a communications adapter 614, a user interface adapter 616, and a display adapter 622. The I/O adapter 610 and/or the user interface adapter 616 may, in certain embodiments, enable a user to interact with the computer system 600. In a further embodiment, the display adapter 622 may display a graphical user interface (GUI) associated with a software or web-based application on a display device 624, such as a monitor or touch screen.

The I/O adapter 610 may couple one or more storage devices 612, such as one or more of a hard drive, a solid state storage device, a flash drive, a compact disc (CD) drive, a floppy disk drive, and a tape drive, to the computer system 600. According to one embodiment, the data storage 612 may be a separate server coupled to the computer system 600 through a network connection to the I/O adapter 610. The communications adapter 614 may be adapted to couple the computer system 600 to the network 110, which may be one or more of a LAN, WAN, and/or the Internet. The communications adapter 614 may also be adapted to couple the computer system 600 to other networks such as a global positioning system (GPS) or a Bluetooth network. The user interface adapter 616 couples user input devices, such as a keyboard 620, a pointing device 618, and/or a touch screen (not shown) to the computer system 600. The keyboard 620 may be an on-screen keyboard displayed on a touch panel. Additional devices (not shown) such as a camera, microphone, video camera, accelerometer, compass, and or gyroscope may be coupled to the user interface adapter 616. The display adapter 622 may be driven by the CPU 602 to control the display on the display device 624. Any of the devices 602-622 may be physical and/or logical.

The applications of the present disclosure are not limited to the architecture of computer system 600. Rather the computer system 600 is provided as an example of one type of computing device that may be adapted to perform the functions of a smartphone 101 and/or the defibrillator 100. For example, any suitable processor-based device may be utilized including, without limitation, personal data assistants (PDAs), tablet computers, smartphones, computer game consoles, and multi-processor servers. Moreover, the systems and methods of the present disclosure may be implemented on application specific integrated circuits (ASIC), very large scale integrated (VLSI) circuits, state machine digital logic-based circuitry, or other circuitry.

The embodiments described herein are implemented as logical operations performed by a computer. The logical operations of these various embodiments of the present invention are implemented (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules. As such, persons of ordinary skill in the art may utilize any number of suitable electronic devices and similar structures capable of executing a sequence of logical operations according to the described embodiments. For example, the computer system 800 may be virtualized for access by multiple users and/or applications.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

1. A mobile defibrillator comprising:
a charging circuit, the charging circuit comprises:
a control sequencer;
a power source;
a charge storage device;
a charge triggering switch coupled to the charge storage device;
a communications interface configured to communicatively couple the control sequencer to a mobile computing device;
a charge driver coupled between the power source and the charge storage device;
a charge sensor coupled between the charge storage device and the control sequencer; and
a signal interface coupled between the charge triggering switch and the control sequencer; and
a pair of adhesive electrodes coupled to the charge triggering switch and the charge storage device.

2. The defibrillator claim 1, wherein
the communications interface comprises a wired communications interface.

3. The defibrillator of claim 1, wherein the communications interface comprises a wireless communication interface.

4. The defibrillator of claim 1, wherein the charge storage device comprises a capacitor.

5. The defibrillator of claim 1, further comprising the charge triggering switch configured to discharge the charge storage device across the pair of adhesive electrodes when the charge trigger switch is transitioned to a closed position.
